(19) United States
(12) Patent Application Publication
(10) Pub. No.: US 2026/0216008 A1
(43) Pub. Date: Jul. 30, 2026

(54) HYGIENE COMPOSITION DISPENSABLE USING APPARATUS

(71) Applicant: Roller Deo Limited, London (GB)

(72) Inventor: Milo Pinckney, London (GB)

(21) Appl. No.: 19/148,763

(22) PCT Filed: Jan. 17, 2024
(86) PCT No.: PCT/IB2024/050443

§ 371 Date: Jul. 17, 2025

(30) Foreign Application Priority Data
Jan. 17, 2023 (GB) 2300669.5

(51) Int. Cl. A61K 8/02; A61K 8/04; A61Q 15/00; A61Q 17/04

(52) U.S. Cl. CPC A61K 8/022; A61K 8/04; A61Q 15/00; A61Q 17/04; A61K 2800/874

(57) ABSTRACT

Disclosed is a formation of a hygiene composition dispensable using an apparatus. The hygiene composition comprises a solute in amount between 4% w/w to 30% w/w with respect to total weight of hygiene composition, wherein solute comprises at least one preservative agent in amount between 0.5% w/w to 3% w/w with respect to total weight of solute, and at least one antimicrobial agent in amount between 5% w/w to 25% w/w with respect to total weight of solute and water in amount between 70% w/w to 96% w/w with respect to total weight of hygiene composition, and wherein the solute is engineered to be mixed and activated upon contact with the water in the apparatus, wherein solute is poured into container of apparatus and water is added into container, wherein apparatus is shaken in order to form hygiene composition having mixture of solute and water.

### TECHNICAL FIELD

This invention relates to fluid compositions used in natural personal care products. In particular, though not exclusively, this invention relates to a formation of a hygiene composition dispensable using an apparatus.

### BACKGROUND

In recent times, the market size of global natural personal care products has been expanding day by day. One of the primary factors that drive the market size expansion is growing awareness about the adverse effects of chemicals on human health. For example, the natural personal care product is an antiperspirant, a lotion, a cleaning material, and so forth.

Notably, the composition of existing natural personal care products includes synthetic preservatives that decrease the naturalness of such products. Moreover, the composition of such products could only be mixed industrially, thereby making such products convenient for only single use. Additionally, the composition of such products is difficult to mix in an application device thereof. For example, the application device is a container, a bottle, and so forth. Furthermore, there are some natural personal care products whose composition fails to disperse evenly within the application device thereof.

Conventionally available natural personal care products include the composition having a wax material, meaning that the entirety of the composition requires shipping. Moreover, such a composition requires mixing outside of the application device using special equipment. However, even then, the composition fails to disperse fully and create a uniform product for final use. In such a case, the ingredients present in the composition settle at a bottom of the application device at the end of the use cycle, thereby reducing the efficacy of such natural personal care products.

Therefore, in light of the foregoing discussion, there exists a need to overcome the aforementioned drawbacks associated with existing natural personal care products.

### SUMMARY OF THE DISCLOSURE

In a first aspect, an embodiment of the present disclosure provides a formation of a hygiene composition dispensable using an apparatus, the hygiene composition comprising:
a solute in an amount between 4% w/w to 30% w/w with respect to the total weight of the hygiene composition, wherein the solute comprising
   at least one preservative agent in an amount between 0.5% w/w to 3% w/w with respect to the solute, and
   at least one antimicrobial agent in an amount between 5% w/w to 25% w/w with respect to the total weight of the solute; and
water in an amount between 70% w/w to 96% w/w with respect to the total weight of the hygiene composition, wherein the solute is engineered to be mixed and activated upon contact with the water in the apparatus, wherein the solute is poured into a container of the apparatus and the water is added into the container, wherein the apparatus is shaken in order to form the hygiene composition having a mixture of the solute and the water.

Throughout the present disclosure, the term “hygiene composition” as used herein refers to a substance (such as a liquid, a gas, and the like) that possesses an ability of continuous deformation thereof under an applied shear stress, or external force. Optionally, the hygiene composition is a mixture of a solute such as a powder concentrate and a solvent such as water. It will be appreciated that the hygiene composition is an efficient, eco-friendly and an evenly distributed composition that could be dispensed using the apparatus. Herein, the term “formation” refers to a method of creating the hygiene composition that is dispensable using the apparatus. It will be appreciated that the solute is engineered to mix, get activated upon contact with the water and uniformly disperse within the apparatus without the need for an external tool or a temperature change. Beneficially, the technical effect of the aforementioned concentration range of the solute is to provide a uniform, usable mixture within the apparatus with the addition of the water added to it in situ. Moreover, said mixture is achieved by pouring the solute into a container followed by the addition of the water, and subsequent shaking to produce the hygiene composition. In an example, the solute is packed in a sachet and whenever required a sachet is torn out in order to add the solute that is packed therein into the container. Subsequently, the water is added to the container. Thereafter, the apparatus is shaken in order to form the hygiene composition having the mixture of the solute and the water. A significant advantage of the formation is that the solute is dissolved readily with the addition of the water in the apparatus, to create a uniform and evenly distributed hygiene composition.

The aforementioned concentration range of the solute ensures the inclusion of a sufficient amount of the at least one preservative agent within the solute. The preservatives when added in the aforementioned concentration range, prevent microbial growth and spoilage, extending the shelf life of the hygiene composition. The aforementioned concentration range of the antimicrobial agent is significant for its role in providing the hygiene composition with antimicrobial properties. This ensures effective protection against potentially harmful microorganisms, contributing to the overall efficacy of the hygiene composition.

The aforementioned concentration of water serves as the medium for activating and mixing the solute. In this regard, the solute is added into the apparatus's container and mixed with the water to allow the formation of the hygiene composition. Optionally, the water could be added first and the solute thereafter in the apparatus. Additionally, the water content facilitates the hydration of the solute, aiding in the dissolution and dispersion of the at least one preservative and the at least one antimicrobial agent. The aforementioned concentrations of the solute and the water contribute to the creation of a homogeneous mixture, preventing uneven distribution of the at least one preservative and the at least one antimicrobial agent. This is vital for consistent performance of the hygiene composition and the user experience. Advantageously, the aforementioned concentration range of the solute and the water provides a desired viscosity to the hygiene composition. The desired viscosity prevents the spillage of the hygiene composition, while in use.

Optionally, the hygiene composition is at least one of: a cosmetic product, a cleaning product, a bleaching product, a polishing product, or a medication. In this regard, the hygiene composition could be any cosmetic, cleaning, bleaching, polishing or medication product that has a final application in the form of the hygiene composition in which the main ingredient is water. It will be appreciated that the hygiene composition is held in the apparatus that facilitates application of the hygiene composition on a surface. Examples of the surface include, but are not limited to, skin, wood, plastic, metal, cloth, leather, and stone. In an example, the apparatus facilitates application of deodorant on skin. In another example, the apparatus facilitates application of topical medication on an infected area of skin.

In this regard, the hygiene composition is prepared by dissolving or diluting a solute into the water. The term “solute” as used herein refers to a substance that is produced by removing or reducing a diluting agent or by selective accumulation of atoms or molecules of the substance. It will be appreciated that the solute is capable of dissolving or dispersing thoroughly in the water without requiring any external tool for the dissolution thereof. Optionally, the apparatus may be shaken or left undisturbed for a certain amount of time in order to increase the rate of dissolution. Optionally, the apparatus may be shaken for the time between 5 seconds to 20 seconds or left undisturbed for the time of 20 to 30 minutes.

Optionally, the solute is in at least one of: a solid state or a liquid state. In this regard, the solute that is dissolved in the hygiene composition may be in the solid state. Examples of the solid state include a powder form, a tablet form, and the like. Optionally, the solute is a powder concentrate. Herein, the term powder concentrate refers to fine, dry particles that are produced by grinding, crushing, and disintegration of a solid substance in order to increase the strength or proportion of the hygiene composition. Optionally, the powder concentrate may be dissolved in a warm water in order to increase the rate of dissolution. Optionally, the amount of the powder concentrate in the apparatus may be up to 30% of the total amount of the hygiene composition. Optionally, the solute that is dissolved in the hygiene composition may be in the liquid state. Examples of the liquid state include a gel form, an emulsion form, an oil form, and the like. Beneficially, the solute comprises various ingredients that are natural and non-toxic. Beneficially, each of the ingredients is added in a pre-defined concentration in order to form the powder thereof. Advantageously, the solute could be dissolved readily with the addition of water in the apparatus, to create a uniform and evenly distributed hygiene composition. In an embodiment, the hygiene composition comprises 4% w/w solute and 96% w/w water with respect to the total weight of the hygiene composition. In another embodiment, the hygiene composition comprises 30% w/w solute and 70% w/w water with respect to the total weight of the hygiene composition. It will be appreciated that the solute is engineered or formulated using a combination of ingredients in accurate proportions such that when the solute comes in contact with the water in the apparatus it forms a uniform hygiene composition inside the apparatus.

The solute comprises at least one preservative agent. Herein, the term “preservative agent” refers to a substance that is added to the hygiene composition to prevent decomposition by microbial growth or by undesirable chemical changes. Optionally, the concentration of the preservative agent lies in the range between 0.5%, 1%, 1.5% or 2% up to 1%, 1.5%, 2% or 3% w/w with respect to the total weight of the solute.

Optionally, the at least one preservative agent is a sodium benzoate. The sodium benzoate is the sodium salt of benzoic acid and appears as a white crystalline chemical with the formula C₆H₅COONa. The sodium benzoate is used as a bacteriostatic and a fungistatic. Optionally, the concentration of the sodium benzoate lies in the range between 0%, 1% or 2% up to 1%, 2% or 3% w/w with respect to the total weight of the solute. Optionally, the at least one preservative agent includes other salts of benzoic acid such as sodium, potassium, or calcium salts to inhibit the growth of bacteria.

The solute comprises at least one antimicrobial agent in an amount between 5% w/w to 25% w/w with respect to the total weight of the solute. Typically, the solute also includes the antimicrobial agents such as antibacterial agents, antifungal agents, and so forth. In this regard, the antibacterial agents are used to treat bacterial infections. The antifungal agents are used to treat fungal infections. Optionally, the concentration of the at least one antimicrobial agent lies in the range between 5%, 10%, 15%, or 20% up to 10%, 15%, 20% or 25% w/w with respect to the total weight of the solute.

Optionally, the at least one antimicrobial agent is selected from at least one of: a sodium anisate, a potassium alum, a leuconostoc/radish root ferment filtrate or a zinc oxide. Optionally, the at least one antimicrobial agent is a sodium anisate. The sodium anisate is a natural preservative obtained from anise and fennel. Beneficially, the sodium anisate possesses a great mitotic inhibitor activity without altering the integrity, colour or pH of other natural ingredients included in the solute. Optionally, the concentration of the sodium anisate lies in the range between 0%, 0,1%, 0.2%, 0.3% or 0.4% up to 0,1%, 0.2%, 0.3%, 0.4% or 0.5% w/w with respect to the total weight of the solute.

Optionally, the at least one antimicrobial agent is a potassium alum. Herein, the potassium alum refers to a chemical compound or the double sulfate of potassium and aluminium, with chemical formula KAI(SO₄)₂·12H₂O. It will be appreciated that the potassium alum inhibits the growth of the bacteria responsible for the odour of the surface. Optionally, the concentration of the potassium alum lies in the range between 0%, 1%, 2%, 3%, 4% or 5% up to 2%, 3%, 4%, 5% or 6% w/w with respect to the total weight of the solute.

Optionally, the at least one antimicrobial agent is a leuconostoc/radish root ferment filtrate. The term “leuconostoc/radish root ferment filtrate” as used herein refers to an ingredient that is extracted as a filtrate obtained from a fermentation of radish root along with the Leuconostoc bacteria. Optionally, the leuconostoc/radish root ferment filtrate ranges in colour from clear to a light amber. Typically, the leuconostoc bacteria and radish root ferments add an antimicrobial peptide that regulates bacteria growth and increases hydration of the skin. Moreover, the leuconostoc/radish root ferment filtrate is a natural antimicrobial preservative and is useful when used topically in products for skin or scalp conditions as it moisturizes and conditions. Furthermore, the leuconostoc/radish root ferment filtrate is water-soluble, anti-fungal, antimicrobial and compatible with a wide range of skincare ingredients. Optionally, the leuconostoc/radish root ferment filtrate could be used in moisturizers, creams, lotions, facial cleansers, shampoos, conditioners, and so forth. Optionally, the concentration of the leuconostoc/radish root ferment filtrate lies in the range between 0%, 1%, 2%, 3%, or 4% up to 2%, 3%, 4%, or 5% w/w with respect to the total weight of the solute.

Optionally, the at least one antimicrobial agent is a zinc oxide. Optionally, the zinc oxide is used as a cosmetic colorant and ultraviolet radiation absorber. Optionally, the zinc oxide is used in the solute for skin protection. Optionally, the concentration of the zinc oxide lies in the range between 0%, 1%, 2%, 3%, 4%, 5%, 6%, 7%, 8%, 9%, 10%, 11%, 12%, 13% or 14% up to 1%, 2%, 3%, 4%, 5%, 6%, 7%, 8%, 9%, 10%, 11%, 12%, 13%, 14% or 15% w/w with respect to the total weight of the solute. Optionally, the at least one inorganic compound is selected from at least one of: sodium bicarbonate, kaolin, magnesium aluminium silicate, magnesium hydroxide, mica, magnesium stearate, silica, and so forth. Optionally, the at least one antimicrobial agent is selected from at least one of: caprylic acid, potassium sorbate, sorbic acid, benzyl alcohol, gluconolactone, benzoic acid, hexanediol, sodium gluconate, lonicera japonica, lonicera caprifolium, and so forth.

Optionally, the solute further comprises at least one texturizing agent in an amount between 5% w/w to 25% w/w with respect to the total weight of the solute. The term “at least one texturizing agent” refers to an additive that is used to enhance the consistency and the touch of the hygiene composition. In an example, palm oil and shea butter are natural texturizing agents. Optionally, the concentration of the at least one texturizing agent lies in the range between 5%, 10%, 15%, or 20% up to 10%, 15%, 20% or 25% w/w with respect to the total weight of the solute.

Optionally, the at least one texturizing agent is selected from at least one of: a trilaurin, a diatomaceous earth, a maranta arundinacea or an Oryza sativa starch. Optionally, the at least one texturizing agent comprises a trilaurin. Herein, the term trilaurin refers to a triglyceride obtained by formal acylation of the three hydroxy groups of glycerol by lauric (dodecanoic) acid. Typically, the trilaurin is a triglyceride and a dodecanoate ester. Optionally, the concentration of the trilaurin lies in the range between 0%, 1%, 2%, 3%, 4%, 5%, 6%, 7%, 8% or 9% up to 1%, 2%, 3%, 4%, 5%, 6%, 7%, 8% 9% or 10% w/w with respect to the total weight of the solute.

Optionally, the at least one texturizing agent comprises a diatomaceous earth. Herein, the term diatomaceous earth refers to a naturally occurring, soft, siliceous sedimentary rock that can be crumbled into a fine white to off-white powder. It will be appreciated that the diatomaceous earth possesses a natural ability to kill parasites, thereby used for making skin exfoliators, scrubs, toothpaste, and so forth. Moreover, the diatomaceous earth removes out the toxins in the skin, making it smooth and supple. Optionally, the concentration of the diatomaceous earth lies in the range between 0%, 5%, 10%, 15%, or 20% up to 5%, 10%, 15%, 20% or 25% w/w with respect to the total weight of the solute.

Optionally, the at least one texturizing agent comprises a maranta arundinacea. The term “maranta arundinacea” (namely arrowroot) as used herein refers to a large, perennial herb found in rainforest habitats. The maranta arundinacea includes an underground edible stem called rhizome. The rhizome comprises multiple roots that store starch and energy. Typically, the rhizome is processed into a fine, white arrowroot powder form (namely arrowroot flour). It will be appreciated that the arrowroot powder possesses various vitamins and minerals and is used as a natural remedy for various skin conditions. Moreover, the maranta arundinacea is used for exfoliating and improving the skin tone. Furthermore, the maranta arundinacea is used for soothing sunburns, rashes, and other skin irritations. Furthermore, the maranta arundinacea is used for absorbing excess oil, softening the skin and reducing inflammation. Optionally, the concentration of the maranta arundinacea lies in the range between 0%, 5%, 10%, 15%, or 20% up to 5%, 10%, 15%, 20% or 25% w/w with respect to the total weight of the solute.

Optionally, the at least one texturizing agent comprises an Oryza sativa starch. The term “Oryza sativa starch” as used herein refers to a high-polymeric carbohydrate material derived from the peeled seeds of rice. It will be appreciated that the Oryza sativa starch is used as an absorbent, a binding agent, and a bulking agent. Optionally, the concentration of the Oryza sativa starch lies in the range between 0%, 1%, 2%, 3%, 4%, 5%, 6%, 7%, 8%, 9%, 10%, 11%, 12%, 13% or 14% up to 1%, 2%, 3%, 4%, 5%, 6%, 7%, 8%, 9%, 10%, 11%, 12%, 13%, 14% or 15% w/w with respect to the total weight of the solute. Optionally, the at least one texturizing agent is selected from at least one of: tragacanth, carrageenan, chitosan, calcium carbonate, charcoal, glycol stearate, and so forth.

Optionally, the solute further comprises at least one thickening agent in an amount between 1% w/w to 10% w/w with respect to the total weight of the solute. Herein, the thickening agent refers to a substance that is used for increasing the viscosity of the hygiene composition without substantially changing other properties thereof. Beneficially, the at least one thickening agent is used to enhance the stability and the overall performance of the hygiene composition. It will be appreciated that thickeners are derived from varied natural sources. Optionally, the concentration of the at least one thickening agent lies in the range between 1%, 2%, 3%, 4%, 5%, 6%, 7%, 8% or 9% up to 2%, 3%, 4%, 5%, 6%, 7%, 8% 9% or 10% w/w with respect to the total weight of the solute.

Optionally, the at least one thickening agent is selected from at least one of: a xanthan gum, a sclerotium gum and an inulin. Herein, the term xanthan gum refers to a polysaccharide that is produced from simple sugars using a fermentation process and derives its name from the species of bacteria such as Xanthomonas campestris. Optionally, the xanthan gum may be used as an effective emulsifier and stabilizer, thus preventing other ingredients of the solute from separating. Optionally, the xanthan gum is used in cosmetics to prepare water gels. Optionally, the concentration of the xanthan gum lies in the range between 0%, 0.5%, 1% or 1.5% up to 0.5%, 1%, 1.5% or 2% w/w with respect to the total weight of the solute.

Herein, the term sclerotium gum refers to a naturally derived, gel-like polysaccharide ingredient that is produced from the fermentation of a fungus such as Sclerotium rolfsii. In this regard, the sclerotium gum is used as a skin conditioning agent. Optionally, the concentration of the sclerotium gum lies in the range between 0%, 0.5%, 1% or 1.5% up to 0.5%, 1%, 1.5% or 2% w/w with respect to the total weight of the solute. Optionally, the at least one thickening agent is selected from at least one of: hydroxyethylcellulose, konjac gum, hyaluronic acid, and so forth.

Herein, the term inulin refers to a group of naturally occurring polysaccharides that are produced by plants such as chicory. It will be appreciated that the inulin is used for thickening the solute when dissolved in the water to form the hygiene composition. Optionally, the concentration of the inulin lies in the range between 0%, 1%, 2%, 3%, 4%, 5%, 6%, 7%, 8% or 9% up to 1%, 2%, 3%, 4%, 5%, 6%, 7%, 8% 9% or 10% w/w with respect to the total weight of the solute. Optionally, the at least one thickening agent is a tapioca starch, a guar gum, a caesalpinia gum, a castor wax, a cetyl palmitate, a lanolin alcohol, a myristyl myristate, a palmitic acid, a polyamide 3, a polymethylsilsesquioxane, a tribehenin, a xylitol, a fructose, a glucose, a glucomannan, a rhamnose, a fructo-oligosacchariderth.

Optionally, the at least one thickening agent is an aloe barbadensis leaf juice. Herein, the term “aloe barbadensis leaf juice” as used herein refers to a succulent plant species of the genus Aloe. It will be appreciated that the aloe barbadensis leaf juice comprises polysaccharides, anthraquinones, phytosterols and glycosides. Typically, the polysaccharides provide Aloe its hydrating, emollient and anti-inflammatory benefits, while creating a protective barrier on the skin. The anthraquinones provide Aloe antimicrobial and antioxidant characteristics. The phytosterols calm and soothe itchy skin, also moisturizes and protects the skin from trans-epidermal water loss. The glycosides promote healthy cell regeneration and offer antihistaminic (namely, anti-allergen) properties, while vitamins, minerals and essential amino acids nourish the skin. Optionally, the concentration of the aloe barbadensis leaf juice lies in the range between 0%, 1%, 2%, 3%, or 4% up to 2%, 3%, 4%, or 5% w/w with respect to the total weight of the solute. Optionally, the at least one plant product is selected from at least one of: zea mays, bentonite, bismuth oxychloride, boron nitride, chamomile hydrosol, calendula, a xylo-oligosaccharide, and so forth.

Optionally, the solute further comprises at least one emollient, at least one anti-inflammatory substance, at least one astringent, at least one viscosity modifier, at least one absorbent, at least one conditioning agent, at least one probiotic complex, and at least one prebiotic complex. Herein, the term emollient refers to a composition that is used for softening the skin. Herein, the term anti-inflammatory substance refers to a substance that reduces inflammation (such as redness, swelling, and pain) in the body of an organism. Optionally, the anti-inflammatory substance is selected from at least one of: Hamamelis virginiana L. (namely, witch hazel), Optionally, the at least one conditioning agent is selected from at least one of: a wasabi ferment, a capryl glycol, and so forth. Herein, the term astringent refers to a substance that shrinks or constricts body tissues. Herein, the term probiotics refers to substances that encompass an array of microorganisms (namely, microbiome) living within and on the surface of the body. In this regard, the solute further comprises the aforementioned ingredients in order to increase the efficacy of the hygiene composition.

Optionally, the solute further comprises at least one prebiotic complex. The prebiotic complex refers to compounds in food that induce the growth or activity of beneficial microorganisms such as bacteria and fungi. The solute includes the prebiotic complex to protect the skin barrier. The strong skin barrier ensures protection against UV damage, environmental pollution, and so forth. Optionally, the concentration of the at least one prebiotic complex lies in the range between 0%, 0.5%, 1%, 1.5%, 2% or 2.5% up to 0.5%, 1%, 1.5%, 2%, 2.5% or 3% w/w with respect to the total weight of the solute.

Optionally, the at least one prebiotic complex is an alpha-glucan oligosaccharide. The term “alpha-glucan oligosaccharide” as used herein refers to an oligosaccharide formed by glucose units that are obtained from the natural sugars sucrose and maltose is commonly found in bacteria, yeasts, plants, and insects. It will be appreciated that the alpha-glucan oligosaccharide act as an emollient and helps increase water retention. Optionally, the at least one prebiotic complex is selected from at least one of: lactobacillus ferment, lactobacillus pentosus, micrococcus kristinae, corynebacterium xerosis, staphylococcus capitis, galactomycesferment, saccharomyces lysate extract, ultrasomes micrococcus lysate, bifida ferment lysate, palmitoyl glycine, and so forth.

Optionally, the solute further comprises at least one aroma compound. In this regard, the solute further comprises the aroma compounds in order to add variety and provide effective protection against the odour of the body. For example, the aroma compounds may form naturally or biochemically during the ripening of fruits and other crops.

Throughout the present disclosure, the apparatus refers to an equipment that allows a regulated dispensation of the hygiene composition while avoiding spills. In this regard, the apparatus is a refillable equipment and thus could be reused several times. Beneficially, the apparatus is environmentally friendly and cost-efficient. It will be appreciated that such apparatus is unique in reducing spillage and leakages of the hygiene composition when not in use to avoid undue wastage and ensure elongated life.

In this regard, the apparatus comprises a container, a cradle and a lid. Optionally, the container is closed at one end such that the hygiene composition does not leak therein. Optionally, the container is a combination of one or more shapes of: a cylindrical shape, an elliptical prism shape, a hexagonal prism shape, an octagonal prism shape. Optionally, the container is open at another end to allow attachment with the cradle and the spherical ball such that the hygiene composition is dispensed in a regulated manner. Herein, the term “cradle” refers to a mechanism which snugly (and/or firmly) holds a spherical ball, such that the spherical ball is movable about the cradle. Herein, the movement of the spherical ball about the cradle is at least one of: a rotational movement, a translational movement. With respect to the rotational movement, the spherical ball rotates about the cradle akin to a roller-ball mechanism to dispense the hygiene composition onto the surface. With respect to the translational movement, the spherical ball may be pushed downward inside the cradle or may move upwards for dispensing the hygiene composition. It will be appreciated that the movement of the spherical ball about the cradle is substantially the rotational movement. It will be appreciated that the cradle is used in the apparatus to dispense the hygiene composition, wherein the hygiene composition coats the spherical ball and is dispensed onto the surface when the spherical ball is moved. In an example, the apparatus facilitates application of deodorant on skin. In another example, the apparatus facilitates topical medication on an infected area of skin. In yet another example, the apparatus facilitates application of application of ink on a paper. Herein, the lid is configured to retain the hygiene composition within the container, wherein the cradle and the lid are removably attached to the container. Notably, such an arrangement forms the fluidic barrier which prevents leakage of the hygiene composition. Moreover, such an arrangement reduces the transportation cost thereof as an entirety of the hygiene composition is not required to be shipped each time.

Optionally, the apparatus may be a spray pump bottle. Typically, the spray pump includes a reservoir, a spraying mechanism and a spray nozzle. It will be appreciated that the spray pump bottle is used for dispensing the hygienic composition. Optionally, the hygienic composition may include a reduced amount of thickening agent when it is dispensed using the spray pump bottle in order to achieve an even distribution thereof on the surface. Optionally, the hygienic composition may include the solute in the amount of 4% w/w with respect to the hygienic composition when dispensed using the spray pump bottle. Optionally, the dispensation of the hygienic composition using the apparatus offers user's a sustainable, convenient, and effective alternative to outdated single-use products and even their refillable counterparts.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the following diagrams wherein:

FIG. 1 illustrates a schematic illustration of an apparatus for dispensing a hygiene composition, in accordance with an embodiment of the present disclosure;

FIG. 2 illustrates a container configured to hold a hygiene composition, in accordance with an embodiment of the present disclosure;

FIG. 3 illustrates a cradle for holding a spherical ball when in use, in accordance with an embodiment of the present disclosure;

FIG. 4 illustrates a lid of an apparatus for dispensing a hygiene composition, in accordance with an embodiment of the present disclosure; and

FIG. 5 illustrates a schematic illustration of the steps of using the apparatus for dispensing the hygiene composition.

### DETAILED DESCRIPTION OF THE DRAWINGS

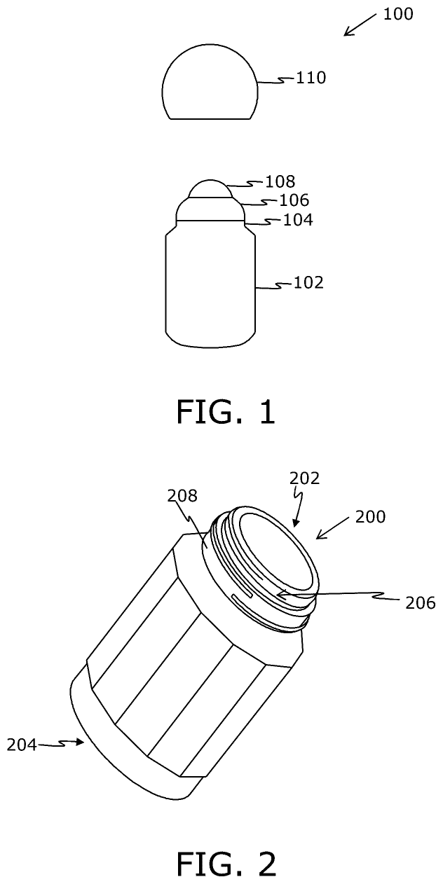

Referring to FIG. 1, illustrated is an apparatus 100 for dispensing a hygiene composition (not shown). The apparatus 100 comprises a container 102 configured to hold the hygiene composition therein. Moreover, the container 102 has a neck portion 104. The apparatus 100 comprises a cradle 106 having a spherical ball 108, wherein when in use the hygiene composition coats the spherical ball and is dispensed onto a surface (not shown) when the spherical ball is moved. Furthermore, the apparatus 100 comprises a lid 110 configured to retain the hygiene composition within the container 102. The cradle 106 and the lid 110 are removably attached to the container 102.

Referring to FIG. 2, illustrated is a container 200 configured to hold a hygiene composition, in accordance with an embodiment of the present disclosure. As shown, the container 200 has a first end 202 which is open and a second end 204 which is closed. A neck portion of the container 200 near (i.e., proximal to) the first end 202 has a first cylindrical portion 206 having a first diameter and a second cylindrical portion 208 having a second diameter. Herein, the second diameter is greater than the first diameter.

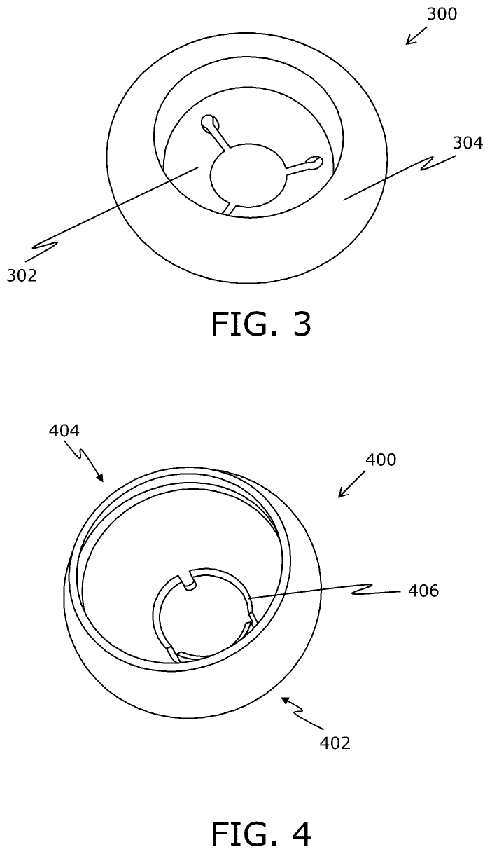

Referring to FIG. 3, illustrated is a cradle 300 for holding a spherical ball when in use, in accordance with an embodiment of the present disclosure. Herein, FIG. 3 illustrates a top-perspective view of the cradle 300. As shown, the cradle 300 comprises a body having a first portion 302 and a second portion 304. As shown, the second portion 304 at least partially covers the first portion 302. The first portion 302 has a hollow hemispherical shape defining a cavity. The hemispherical shape of the first portion of the cradle is advantageous since it allows the spherical ball to be accommodated therein.

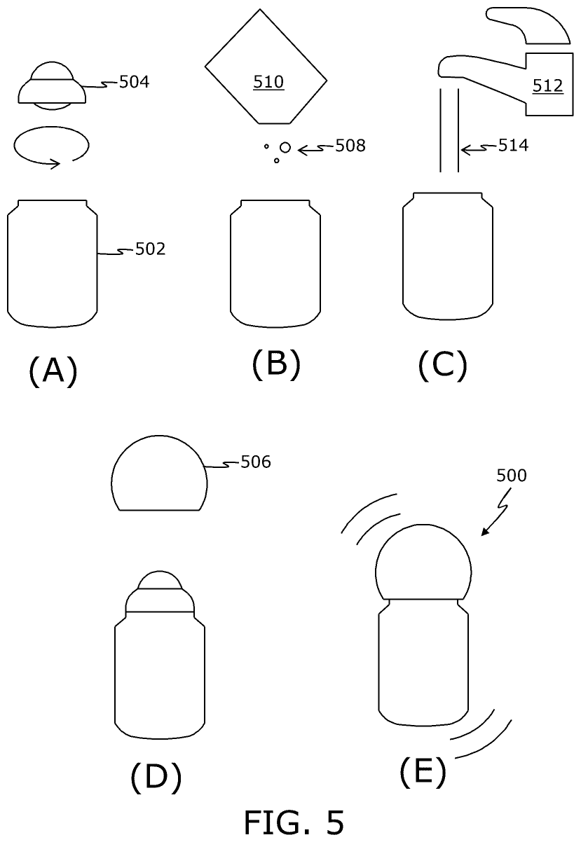

Referring to FIG. 4, illustrated is a lid 400 of an apparatus for dispensing a hygiene composition, in accordance with an embodiment of the present disclosure. The lid 400 comprises a closed end 402 and an open end 404. An inner face of the lid 400 at the closed end 402 has a protruding element 406. When in use, the lid 400 covers a portion of a container capable of holding the hygiene composition, and the protruding element 406 applies an external force on a spherical ball held in a cradle (not shown).

Referring to FIG. 5, illustrated is a schematic illustration of the steps of using an apparatus 500 for dispensing the hygiene composition. As shown, illustrated are components of the apparatus such as a container 502, a cradle 504, a lid 506. Moreover, there is shown a solute 508 that is packed in a sachet 510. Furthermore, there is shown a tap 512 that is used as a source of water 514. At step A, the cradle 504 is unscrewed from the container 502 of the apparatus 500. At step B, the sachet 510 is torn in order to pour the solute 508 that is packed therein into the container 502. At step C, the water 514 is added into the container 502 in order to form the hygiene composition. At step D, the container 502, the cradle 504 and the lid 506 are re-attached with each other. At step E, the apparatus 500 is shaken for a few seconds (such as 20 seconds) in order to form a uniform hygiene composition having a mixture of the solute 508 and the water 514.

FIGS. 1, 2, 3, 4 and 5 are merely examples, which should not unduly limit the scope of the claims herein. A person skilled in the art will recognize many variations, alternatives, and modifications of embodiments of the present disclosure.

1-11. (canceled)

12. A method of using an apparatus to dispense a hygiene composition,
wherein the apparatus includes:
(i) a container configured to hold the hygiene composition therein, wherein the container includes a neck portion,
(ii) a cradle that has a spherical ball that, when in use, is coated in the hygiene composition, wherein the ball is configured to dispense the hygiene composition when moved, and wherein the cradle is removably attached to the container;
(iii) a lid that is removably attached to the container;
wherein the hygiene composition comprises a solute comprising:
(iv) at least one preservative agent in an amount between 0.5% w/w/to 3% w/w with respect to the total weight of the solute; and
(v) at least one antimicrobial agent in an amount between 5% w/w to 25% w/w with respect to the total weight of the solute;
wherein the method includes:
(a) pouring the solute into the container;
(b) adding water into the container in an amount between 70% w/w/to 96% w/w/with respect to the total weight of the hygiene composition; and
(c) shaking the container including its contents of the solute and the water to mix them to form the hygiene composition.

13. The method of claim 12, wherein the method includes configuring the solute to be in at least one of: a solid state or a liquid state.

14. The method of claim 12, wherein the method includes configuring the at least one preservative agent to be sodium benzoate.

15. The method of claim 12, wherein the method includes configuring the at least one antimicrobial agent to be selected from at least one of: a sodium anisate, a potassium alum, a leuconostoc/radish root ferment filtrate or a zinc oxide.

16. The method of claim 12, wherein the method includes configuring the solute to further comprise:
a least texturizing agent in an amount between 5% w/w to 25% w/w with respect to the total weight of the solute; and
at least one thickening agent in an amount between 1% w/w to 10% w/w with respect to the total weight of the solute.

17. The method of claim 16, wherein the method further includes configuring the at least one texturizing agent to be selected from at least one of: a trilaurin, a diatomaceous earth, a maranta arundinacea or an Oryza sativa starch.

18. The method of claim 16, wherein the method includes configuring the at least one thickening agent to be selected from at least one of: a xanthan gum, a sclerotium gum, and an inulin.

19. The method of claim 12, wherein the method includes configuring the solute to further comprise a least one emollient, at least one anti-inflammatory substance, at least one astringent, at least one viscosity modifier, at least one absorbent, at least one conditioning agent, at least probiotic complex, and at least one prebiotic complex.

20. The method of claim 12, wherein the method includes configuring the hygiene composition to be at least one of: a cosmetic product, a cleaning product, a bleaching product, a polishing product or a medication.
